(19) United States
(12) Patent Application Publication
(10) Pub. No.: US 2023/0204555 A1
(43) Pub. Date: Jun. 29, 2023

(54) USE OF OUTPUT OF REAL TIME SENSORS TO AUTOMATICALLY TRIGGER DEVICES

(71) Applicant: AirAnswers, Inc., North Chicago, IL (US)

(72) Inventors: Julian Gordon, Lake Bluff, IL (US); James J. Koziarz, Highland Park, IL (US)

(21) Appl. No.: 18/090,868

(22) Filed: Dec. 29, 2022

Related U.S. Application Data
(60) Provisional application No. 63/294,583, filed on Dec. 29, 2021.

(51) Int. Cl. G01N 33/00; G01N 1/22

(52) U.S. Cl. CPC G01N 33/0062; G01N 1/2273; G01N 1/26

(57) ABSTRACT

As described herein, a method for measuring air quality comprises operating a real-time air quality sensor to continually measure a parameter of environmental air quality; automatically determining if the parameter indicates that a select environmental condition has been reached and providing an output signal responsive thereto; communicating the output signal to an air-quality sampling system; automatically operating the air-quality sampling system responsive to the output signal to collect an air sample for a pre-determined time; and providing laboratory analysis of the collected air sample.

### CROSS-REFERENCE TO RELATED APPLICATIONS

This application claims priority of Provisional Ser. No. 63/294,583, filed Dec. 29, 2021, the disclosure of which is hereby incorporated by reference in its entirety.

### FEDERALLY SPONSORED RESEARCH OR DEVELOPMENT

Not Applicable.

### MICROFICHE/COPYRIGHT REFERENCE

Not Applicable.

### FIELD OF THE INVENTION

This application relates to air quality and, more particularly, to use of a real time air quality sensor to trigger an air-quality sampling system.

### BACKGROUND OF THE INVENTION

Numerous sensing devices or sensors are available commercially for monitoring ambient air for such parameters as particles of various size ranges, volatile organic compounds (VOC's), carbon dioxide, carbon monoxide, and radon. Sensors are devices which measure potential pollutants or toxic substances in the air.

Temperature and humidity measurements may be required for calibration of values of sensor measurements [1-5], but are not categorized as sensors herein. Sensor measurements are continuously monitored parameters that may be directly or indirectly related to health. Indeed, it has been proposed, but not verified in practice, that given enough experience in correlating symptoms with combinations of these parameters, conditions can be identified, or even predicted, when allergens will trigger asthma or rhinitis. For example, AlerSense Inc. previously used proprietary algorithms and data in order to continually improve sensing conditions in the home. However, no data is available to show how this is borne out in practice.

Sensors may also respond to normally changing conditions which might not be of any consequence. For example, normal cooking activity will release VOCs into the air or toothbrushing will generate particulate aerosols. Air sample collection and analysis may then be required to provide to distinguish between spurious results and presence of a specific toxic substance. Commercially available sensors such as discussed above are not known to be used to trigger a sample collection device to distinguish between toxic and non-toxic substances, or to activate of an air cleaning device.

Air samplers are known in the art that can collect particulates or aerosols for more specific analysis of viruses, bacteria, molds, allergens or other biologically active agents. Examples are air filters, Anderson Impactor, Burkhard spore collector, Innovaprep Bobcat, NIOSH BC-251, Ace Glass AGI-30 and Electrokinetic air sampling devices sold by Applicant and as specified in detail in one or more of U.S. Pat. Nos. 8,038,944; 9,216,421; 9,360,402; 10,245,577; 11,353,499 and 11,275,183. All of these devices have to be run for a predetermined period of time sufficient to detect whatever biological material is collected, the sample has to be collected from the device and then subject to any one of a number of specific tests. Tests may include colony counts for bacteria or molds, plaque counts for viruses, PCR for nucleic acids or organisms containing nucleic acids, limulus amebocyte tests for bacterial endotoxins or mold cell wall fragments, volatile organic compounds, immunoassays for allergens or other substances. All of these tests rely on turning a device on, running it for a predetermined time, collection and processing of sample, and testing sample. These are more powerful and specific measurements but require more skill, are time-consuming, and usually more expensive than direct measurement with real time sensors. None of these devices are known to be automatically triggered by another sensor.

There are numerous reports on the merits of networking sensors. The following review various aspects of sensor technology: Fanti et al [6] reviewed next-generation sensors including mobile phone apps. Hernandez-Gordilla et al [7] have reviewed work on crowd-sensing for pollutants. Zimmerman [8] published guidelines for designing low-cost sensor networks. Specific works describe improvements using networked sensors. Li et al [9] describe a method for improving the quality of data from low-cost particle sensors in a woodworking shop to show dust generation and ventilation and to an extensive ground network compared with remote sensing by satellite [10]. Xiang et al [2] improved sensor accuracy in a network with a remote calibration technique, and improved information with a combination of mobile and stationary sensors [11]. Cho et al [4] also showed the benefits of accurate calibration of a network of multiple sensors and remote calibration. Prakash et al [12] demonstrated the efficacy of a low-cost sensor network providing real-time maps under both high and low pollution conditions. Cowell et al [5] describe a low-cost network of battery-operated devices with correction of values at varying humidities. Arroyo et al [13] describe a networked system with sensors for 5 pollutants at each location showing performance against reference methods. Timonen et al [14] created a network of multi-pollutant sensors and showed the effects of overall weather conditions and traffic conditions. Cabral et al [15] designed a novel mobile handheld GPS-connected particle sensor and showed the utility of data when carried by participants. Similarly, Chatzidiaku et al [16] evaluated a system with particle, NO, NO₂, CO, O₃ sensors in portable devices as part of a networked system. None of the above suggests the use of a networked system to trigger a secondary device or analysis when a critical value has been obtained.

Many air cleaning devices are commercially available where the cleaning is controlled by a built-in particle sensor, but have the disadvantage that the cleaner response is determined by conditions localized to the cleaner. It is more useful for the sensor to respond to values from networked sensors that are in the same location, but closer to the source of pollutants, or at other locations within the same facility under a common HVAC system. An example is a Carrier Smart Air Purifier which enable monitoring and control of the air purifier with a WiFi enabled system, and permits adjustment of control from a remote location. Built-in sensors for particles and VOCs do not sense the air except in the device itself. A disadvantage of all air cleaning devices with sensor control then is that they only sense the air in the immediate vicinity of the device, while pollutants may be building up at other locations within the same facility. Air cleaning devices are specified for the area of the space in which they are installed. This applies to an ideal location, but in practice a gradient of concentrations may be present because of the dynamic interplay between the source of the pollutant and the cleaning device as a sink.

### SUMMARY OF THE INVENTION

As described herein, a method of sensing air quality uses a networked system to trigger a secondary device or analysis when a critical value has been sensed.

In accordance with one aspect, a method for measuring air quality comprises operating a real-time air quality sensor to continually measure a parameter of environmental air quality; automatically determining if the parameter indicates that a select environmental condition has been reached and providing an output signal responsive thereto; communicating the output signal to an air-quality sampling system; automatically triggering the air-quality sampling system responsive to the output signal to collect an air sample for a pre-determined time; and providing laboratory analysis of the collected air sample.

The triggered action may be turning on a more efficient air cleaning system throughout a facility or activating an air sampling system that requires more than an instant reading of a parameter, such as initiating an air sampler that is required to run for a specified time for collection of a sample, that will be subject to more sophisticated analysis, such as by immunological or molecular methods. Having a process automatically triggered will both save manual steps by an operator and provide a speedier reaction to changing circumstances. The process may be monitored remotely on a dashboard so that both the dataset streams from the sensors and the reaction of devices to the values can be observed remotely, and optionally terminated manually when pre-defined critical conditions no longer apply. The dashboard may also be used to monitor the status of multiple devices, including multiple different kinds of device, and also provide for the optional over-ride of an automatically initiated function.

Other objects, features, and advantages of the invention will become apparent from a review of the entire specification, including the appended claims and drawings.

### BRIEF DESCRIPTION OF THE DRAWINGS

FIG. 1 represents a basic configuration of a method for measuring air quality as described herein;

FIG. 2 represents the basic configuration of the method wherein a sensor and triggered system are connected via the internet;

FIG. 3 represents a configuration where the status of the sensor and triggered device may be monitored remotely, and two-way communication exists between the three elements of the system;

FIG. 4 represents a multiplexed configuration using a multiplicity of sensors and a multiplicity of triggered devices;

FIGS. 5A, 5B and FIG. 5C illustrate alternative embodiments of the method showing possible connectivity options between the Triggering Device and the Triggered Device;

FIG. 6A depicts a one-to-one wireless communication between the Triggering Device and the Triggered Device utilizing the Internet;

FIG. 6B depicts a one-to-many wireless communication between the Triggering Device and specifically Triggered Device, or multiple Triggered Devices utilizing the Internet;

FIG. 7 depicts wireless network connectivity between one-or-many Triggering Device(s) and one-or-many Triggered Device(s).

FIG. 8 depicts control and/or monitoring of the Triggering Device and Triggered Device by the user via a Dashboard, all done wirelessly via the Internet;

FIG. 9 illustrates possible hosting devices for user's Dashboard; and

FIG. 10 depicts an embodiment of the invention that encompasses multiple Triggering and Triggered devices capable of wireless Internet connectivity that can be controlled and/or monitored by a user via the Dashboard.

### DETAILED DESCRIPTION OF THE PREFERRED EMBODIMENT

Disclosed herein is a method for using a real-time air quality sensor to trigger an air-quality sampling system, wherein the sampling system collects air samples for a pre-determined time and subsequent laboratory analysis of collected material.

The method herein is advantageous as it automatically triggers the running of a secondary device to eliminate the need for human intervention. The advantage is a greater economy of operation. The secondary device may be an air cleaning device that will be run more economically only when needed and will not over-clean when not needed. The presence of a microbiome in the air may be a requisite of health. Also, complete removal of humidity may result in respiratory disorders. In the case of the secondary device being an analytical device, the likelihood that the analytical procedures will only result in negative result will be reduced. This will be an important economy since the secondary devices are usually the most expensive part of the process. A sample collection device will only run when a specific environmental condition has been reached and will not run when there is a likelihood that the sampler will produce negative results. This is equivalent to screening and confirmatory in diagnostic medicine except that the screening test result, being an electrical signal or signals, can be programmed to trigger the confirmatory without human intervention. Any one or combination of the sensors in Table 1, below, can be used to signal that there is a likelihood of occurrence of a condition where the confirmatory will yield a positive result, thus avoiding unnecessary runs when the result would be negative. For example, high total VOCs could trigger the pump that will collect the VOCs on a charcoal filter for subsequent analysis by mass spectrometry. Mass spectrometry will show whether the toxic compounds are present amongst the VOCs or whether VOCs in characteristic chemical spectra found from mold growth can be found. A predetermined critical particle count from a Dylos particle counter modified with a wireless connection to central control station with a dashboard indication, especially smaller size particles (e.g. PM 0.5), will be indicative that allergens might be present. This could then automatically power on an Electrokinetic air sampling device such as specified in detail in one or more of Applicant's U.S. Pat. Nos. 8,038,944; 9,216,421; 9,360,402; 10,245,577; 11,353,499 and 11,275,183, the specifications of which are hereby incorporated by reference herein. Such a device will run for a preset time to collect an air sample, and the sample collected would be analyzed for suspected allergens, and the specific allergen or allergens, if present, determined by laboratory procedures.

While the use of combinations of sensor results to predict the presence of specific allergens is not established, the use of such data to predict an increased probability of an allergen or allergens being present in the air, will result in a great economy by avoiding running sophisticated sampling devices when the results are going to be negative. Once the triggering condition has been ascertained, it is facile to use the electrical output of the sensor to trigger the running of the sampler without human intervention, thus resulting in greater economy and efficiency than running the sampler under all conditions. The system can be networked to include data on a multiplicity of samplers at different locations with the same facility.

The real-time sensor system may be placed outside and trigger the mentioned Electrokinetic air sampling device when, for example, a high pollen or mold spore count occurs outside and there is a risk of subsequent events indoors. This would be especially important under temperate weather conditions when open windows are used to maintain indoor temperatures at comfortable levels. In a similar manner, additional air cleaning power may be triggered through wirelessly connected communication. Additional cleaning power could be achieved by the fan of an HVAC system being turned to the “on” status independently of the temperature and humidity control of the HVAC system. The HVAC system would then pass the air through its built-in filter system. The HVAC filter may comprise a HEPA filter or an electrostatic precipitation system.

Any one or combination of signals from real-time devices listed in Table 1 may be used as a triggering device as will be apparent to one skilled in the art. For example, particle counts from a Dylos counter may be used to trigger an air sampler, and the sample collected may be subject to a variety of possible assays, including immunoassays or PCR-based assays.

Table 1, below, lists numerous known environmental real-time sensors for measuring different aspects of environmental air quality.

TABLE 1

| Source | Metrics |
|---|---|
| https://www.linkedin.com/in/alersense/ | Temperature, humidity, TVOC, |
| | PM10, noise. |
| http://air-mentor.eu/ | Temperature, humidity, CO2, |
| | TVOC, PM2.5, PM10. |
| http://aircasting.org/ | Temp humidity PM1, PM2.5 |
| | and PM10 |
| https://www.airthings.com/en-us/ | Temperature, air pressure, |
| | humidity, TVOC, and CO₂ |
| https://www.airveda.com/airveda-product-catalo [symbol] | PM2.5, PM10, CO2, |
| | Temperature & Humidity. |
| https://www.alersense.com/product/ | Temperature, humidity, CO2, |
| | TVOCs, large and small particles |
| http://www.alphasense.com/index.php/air/ | CO, H2S, metal oxides, methane, |
| | NO, NO2, O3, PM1, PM2.5, PM10 |
| | (PM4.25 as an option), SO2 |
| https://atmotube.com/ | Temperature, humidity, CO2, |
| | TVOCs, fine dust |
| https://getawair.com/ | Temperature, humidity, CO2, |
| | TVOC, PM1, PM2.5, PM10, |
| | barometer/altimeter |
| http://birdihome.com/tech.html | Temperature, humidity, CO2, CO, |
| | ambient light |
| https://www2.blueair.com/in/ | Temperature, humidity, TVOC, |
| blueair-aware#productTechnicalSpecs | PM2.5 |
| https://www.cairsensors.com/air-quality.html | Temperature, humidity, TVOC, |
| | large and small particles |
| https://cubesensors.com/#design | Temperature, humidity, VOCs, |
| | noise, light, air pressure |
| http://www.dylosproducts.com/ | Temperature, humidity, |
| | PM 0.5, PM2.5 |
| https://www.edimax.com/edimax/merchandise/ | Temperature, humidity, PM2.5, |
| merchandise_detail/data/edimax/global/ | PM10, CO2, TVOC, HCHO, |
| air_quality [symbol] monitoring_semioutdoor/ai-1001w_v3/ | |
| https://www.amazon.in/Pollution-Detector-Particulate- | PM2.5, HCBO, TVOC |
| Formaldehyde-Volatile/dp/B074FTVHFN | |
| https://www.elcomponent.co.uk/air-quality/ | Separate sensors for Temperature, |
| | humidity, PM2.5, PM10, CO2, |
| | TVOC, HCHO, |
| http://www.elitechus.com/product-category/ | PM2.5, HCHO, TVOC, AQI |
| air-quality-detector/ | |
| https://www.familysafetyproductsinc.com/ | Radon |
| https://foobot.io/foobotspecs.pdf | Temperature, humidity, PM2.5, |
| | TVOC |
| http://www.freshairsensor.com/ | Tobacco or marijuana smoke |
| https://www.amazon.com/IGERESS-Measuring- | Temperature, humidity, PM2.5, |
| Formaldehyde-Pollution-Recording/dp/B07L4CXDC9 | PM1, PM10, CO2, TVOC, HCHO, |
| https://learn.kaiterra.com/en/air-academy | PM 2.5, TVOC |
| https://www.golighthouse.com/en/environmental-sensors | Particles 0.3 μm-25 μm |
| https://metone.com/indoor-controlled-environments/ | PM1, PM2.5, PM4, PM10 & TSP |
| indoor-air-monitoring/aerocet-831-handheld-particle-counter/ | |
| https://www.netatmo.com/en-us/aircare/ | Temperature, humidity, |
| homecoach/specifications | CO2, sound |
| http://inovafitness.eom/en/a/chanpinzhongxin/95 [symbol] html | Various PM2.5 testers |
| https://www.onsetcomp.com/products?f%5B0%5 [symbol] D= | Temperature/Humidity, CO2, |
| im_taxonomy_vocabulary_8%3A547&f%5B1%5D= | |
| im_taxonomy_vocabulary_14%3A53 | |
| http://www.plantower.com/en/ | Various PM2.5 testers |
| https://plumelabs.com/en/air/ | Temperature, humidity, NO2, |
| | TVOC, fine particles |
| http://radonftlab.com/radon-sensor-product/rd200/ | Radon |
| http://www.sensology.cn/ | Temperature, humidity, TVOC, |
| | PM2.5, PM1, PM2.5, PM10 |
| https://www.specksensor.com/ | Temperature, humidity, PM2.5 |
| http://www.sprimo.com/ | Temperature, humidity, TVOC |
| https://www.amazon.com/dp/B07DHW98ST/ | HCHO, TVOC |
| ref=dp_cerb_1 | |
| https://uhooair.com/how-it-works/ | Temperature, humidity, |
| | TVOC, CO2, CO, PM2.5, |
| | NO2, O3, air pressure |
| https://pierasystems.com/products | Temperature, humidity, CO2, |
| | air pressure, VOC, particles |
| | in bins PM0.1-PM10 |

[symbol]

indicates data missing or illegible when filed

As described below, sensors such as those referenced in Table 1 may be networked with an Electrokinetic air sampling device or the like for further measurement when required.

In accordance with the methodology provided herein, the real-time sensors continually measure one or more parameters of environmental air quality and automatically determining if the parameter(s) indicates that a select environmental condition has been reached. Moreover, the real-time sensors are equipped with or modified to provide an output signal responsive thereto. The output signal may be a wired signal or a wireless signal, as described below. The output signal is communicated to the triggered device. The triggered device is likewise operative to be actuated in response to receiving the output signal. Once actuated, then the triggered device will operate for a select time to acquire an adequate sample, as is known.

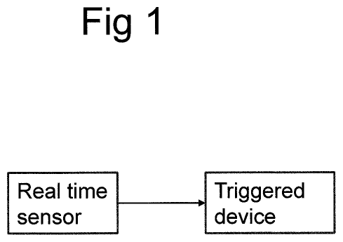

In FIG. 1 there is illustrated an embodiment using direct one-way communication between a real-time sensor and a triggered device. This may be used when the devices are present in the same space and a hardwired connection, or a Bluetooth connection, or the like, will enable the sensor to trigger the device when a critical value of a parameter is exceeded.

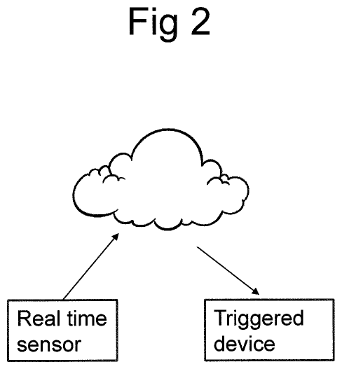

In FIG. 2 there is illustrated an embodiment in which the real-time sensor and the triggered device are networked through the cloud. These devices could use Wi-Fi communications to local network interfaces for communication over the internet. Such communications are generally well known. The device will be triggered by the sensor when a predetermined critical level is determined by the sensor. This will increase the flexibility of location of the sensor within a space without restriction undue restriction of the locations of the sensor or device.

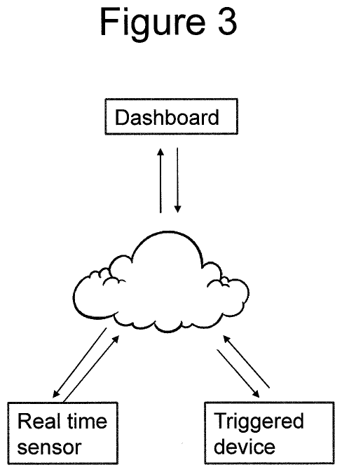

FIG. 3 shows an embodiment further including use of a dashboard in communication with the sensor and the triggered device through the cloud, and further incorporating 2-way communication. This enables a user at a remote location using the dashboard to observe the status of sensor and device, and, optionally to control the function of the device to override the triggering function. The dashboard may advantageously be programmed to provide a history of the statuses of the sensor and the device. This will alleviate the user from having to continuously monitor the status of the device. Calibration history and status of the sensor and re-calibration may be achieved remotely through the dashboard. This will follow conventional methods of re-calibration.

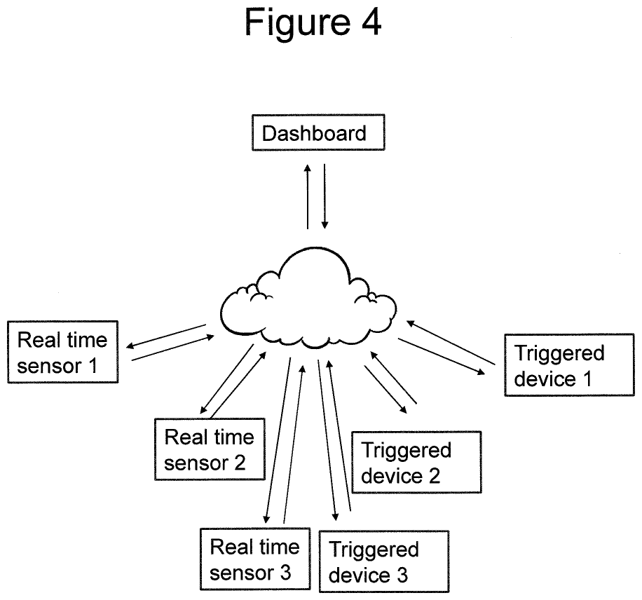

FIG. 4 illustrates an embodiment which increases power of the system and method by including multiple sensors and multiple triggered devices. This permits the monitoring and triggering of devices throughout a room, a facility or even a geographic region such as a neighborhood, a town, a city, a state or even a whole country. The other capabilities described in connection with FIG. 3 will also be available.

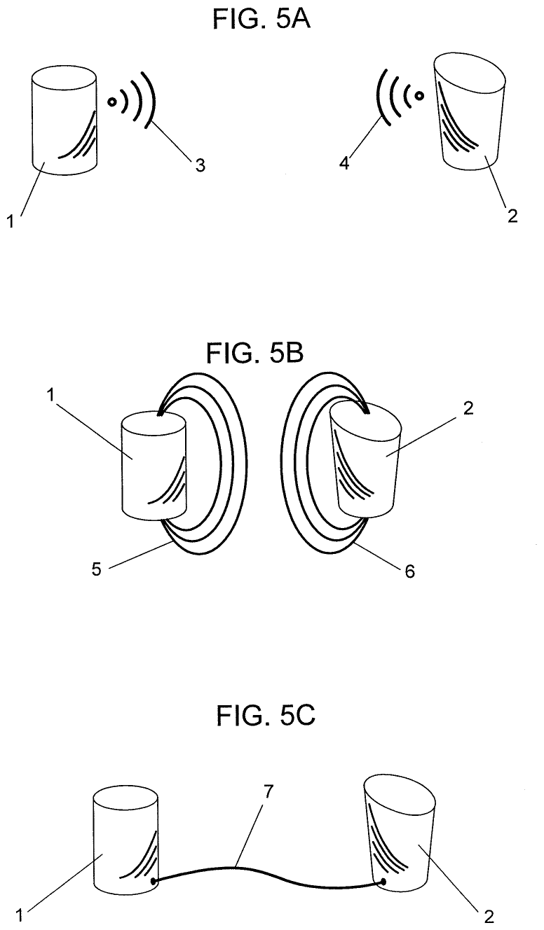

FIGS. 5A, 5B and 5C illustrate alternative embodiments of the invention showing the possible connectivity between the triggering device 1 and the triggered device 2. The triggering device 1 can be an air quality sensing device (sensor) with specific sensing capability(s) operating in real-time. The triggered device 2 can be a more sophisticated air quality sensing or other air contaminant monitoring device with on-board analysis/reporting capability. The triggered device 2 can also be an air quality mitigating device/controller such as for air ventilation, humidity, and ambient or process control.

The device 1 in one embodiment could incorporate both a real time air monitoring sub-system and a sample collection subsystem. Typically, the air monitoring sub-system would be a particle counter and the sample collection sub-system would be and electro-kinetic capture device with no moving parts. In more typical embodiments the air monitoring subsystem in device 1 would be a separate stand-alone sensor system detecting any one of the real-time sensor tests outlined in Table 1 and the prior art description. The air sampling collection sub-system would be a separate stand-alone sampling system in device 2 such as an electrokinetic capture system with no moving parts, or a filter capture system such as a Zephon filtration cartridge as in well-known in the art, with air flow driven by a pump or a fan, or an Andersen or SAS type collector, or impinger or impactor samplers. In all cases the sample is removed from the respective device and transported to a laboratory for chemical, or biochemical analysis. In the case of the capture medium being a solid phase, such as a filter on an electrode, the sample is further extracted as a solid. Where the sampling is in a liquid, the liquid is transported and subject to analysis.

FIG. 5A shows Radio Frequency (RF) wireless communication/control links 3 and 4 between the triggering device 1 and the triggered device 2. A non-exhaustive example would be WiFi or Bluetooth.

FIG. 5B shows Near Field wireless communication/control links 5 and 6 between the triggering device 1 and the triggered device 2. A non-exhaustive example would be RFID.

FIG. 5C shows a hard-wired (cable) communication/control link between the triggering device 1 and the triggered device 2. A non-exhaustive example would be USB, Ethernet, or Serial cable.

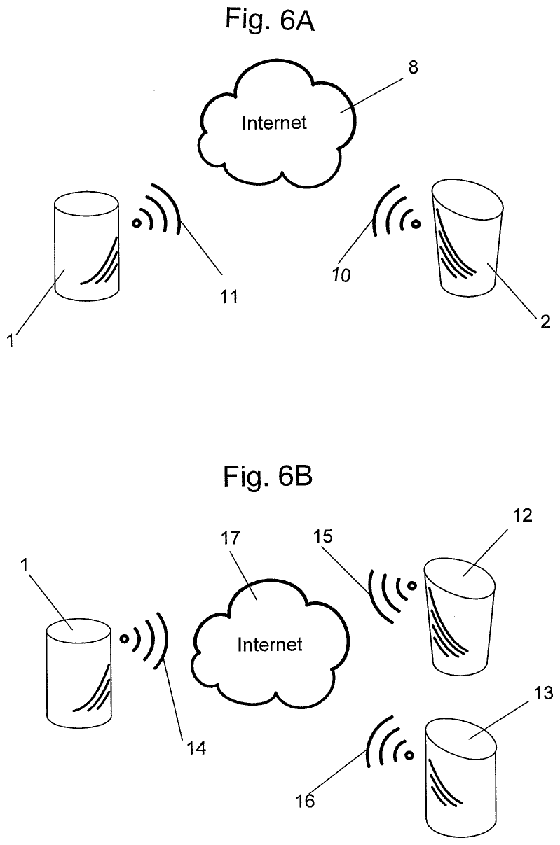

FIG. 6A depicts one-to-one wireless communication via links 11 and 10 between the triggering device 1 and the triggered device 2 utilizing the Internet 8.

FIG. 6B depicts one-to-many wireless communication via links 14, 15 and 16 between the triggering device 1 and specifically one triggered device 12, or multiple triggered devices 12 and 13 utilizing the Internet 17.

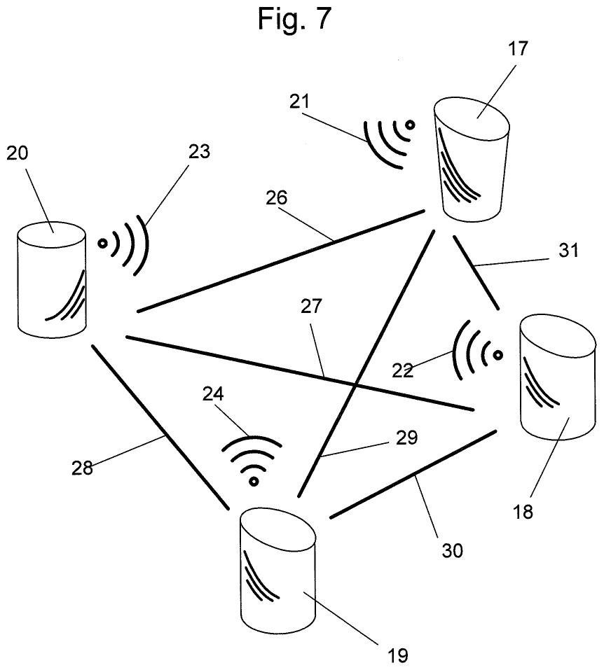

FIG. 7 illustrates four devices 17, 18, 19 and 20 having respective communication modes 21, 22, 44 and 23. As is apparent, more or fewer of such devices may be utilized. These devices 17-20 can be any combination of real-time sensors or triggered devices and each can communicate over any of links 26-31 with each of the other devices, as is shown. This wireless network connectivity can be established between one-or-many triggering device(s) and one-or-many triggering device(s). The network can be established as an ad-hoc (self-organizing connectivity) or mesh network (many-to-many connectivity). This can be done via WiFi or Bluetooth.

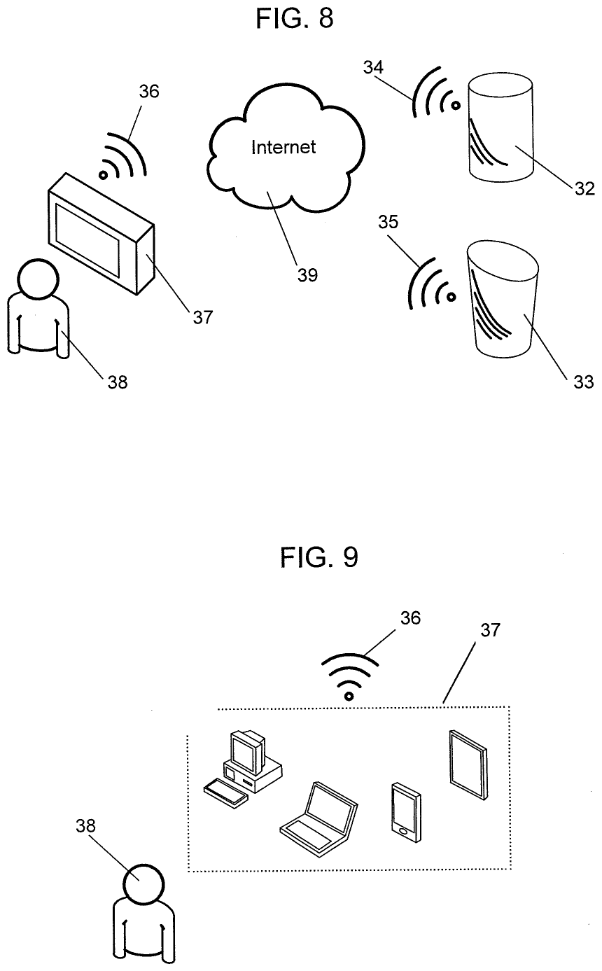

FIG. 8 illustrates wireless communication via links 34 and 35 between the triggering device 32 and the triggered device 33 utilizing the Internet 39. Control/monitoring of the triggering device 32 and the triggered device 33 is overseen by a user 38 using a personal computer or the like programmed as a dashboard 37. The dashboard 37 communications via a link 36 to the Internet 39. The dashboard 37 uses a Graphical User Interface (GUI) that can display monitored Air Quality/Air Contaminants data/information. Using the dashboard 37 the user 38 can also issue commands to the triggering device 32 or the triggered device 33. For example, the triggering device 32 may alert the user 38 to activate the triggered device. The alert would be based upon an environmental change in one or more parameters detected by the triggering device 32.

FIG. 9 illustrates possible hosting devices for the dashboard 37. Devices like a desktop computer, notebook computer or laptop, tablet or smart phone with wireless connectivity capability can run the GUI software to facilitate the dashboard functionality.

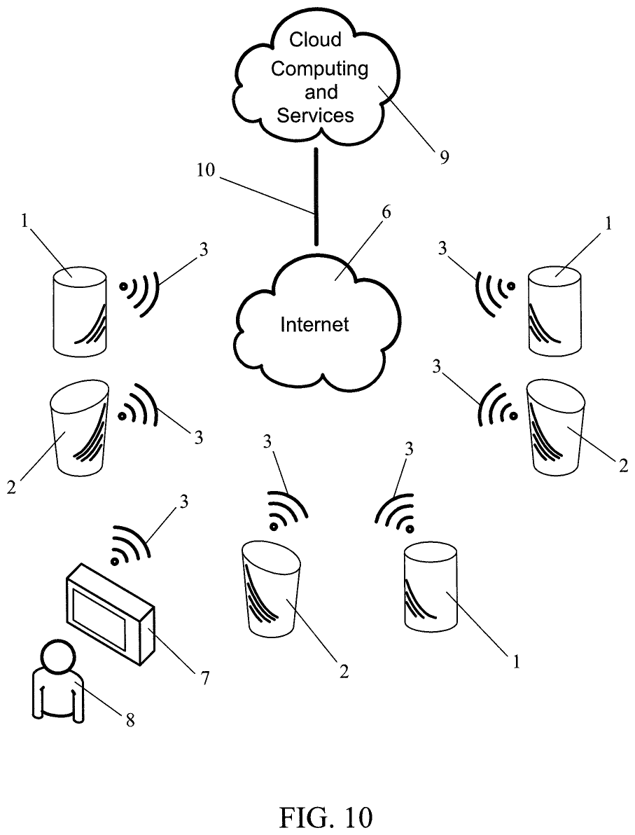

FIG. 10 illustrates an embodiment that encompasses multiple triggering and devices 1 and triggered devices 2 with wireless connectivity via 3 to the Internet 6. These devices can be controlled/monitored by a user 8 via the dashboard 7. This embodiment further includes cloud computing 9 via a link 10 and back-end services to facilitate the collection, processing, and data storage of monitored air quality/air contaminants/mitigation action(s) triggered and/or taken. Additionally, cloud services could be used for the storage and issuance of firmware updates for both triggering and triggered devices.

Thus, there is generally described herein a method for measuring air quality comprising operating a real-time air quality sensor to continually measure a parameter of environmental air quality; automatically determining if the parameter indicates that a select environmental condition has been reached and providing an output signal responsive thereto; communicating the output signal to an air-quality sampling system; automatically triggering the air-quality sampling system responsive to the output signal to collect an air sample for a pre-determined time; and providing laboratory analysis of the collected air sample.

The laboratory analysis may be nucleic acid-based for detection of pathogens, immunologically based for detection of toxins or allergens, or based on limulus amebocyte assays for detection of pyrogens.

The sampling system may collect bio-material over a pre-determined time. for subsequent analysis.

The air quality sensor may be, for example, a particle sensor, a temperature sensor, a humidity sensor, a volatile organic compound sensor, a CO2 sensor, a CO sensor, an H2S sensor, an NO sensor, an NO2 sensor, an HCHO sensor, or a methane sensor.

The air sampling system may also be a Burkart sampler or an Anderson impactor.

### EXAMPLE

| | Dylos counts PM | AirAnswers |
|---|---|---|
| | 0.5 (particles/cu ft) | status |
| Typical background | 200,000 | Off |
| Trigger level | >500,000 | Triggered |

A Dylos particle counter is running continuously as a real-time sensor. The particle count in the air is recorded once per minute in particles up to 0.5 nm (PM 0.5). The counter is connected by Bluetooth to an electrokinetic air sampling device commercialized by the assignee and as discussed above. The “triggered” status is activated at the level indicated. When triggered, the sampling device will continue to run for 24 hrs. A green LED in the sampling device indicates that it is running. When the LED changes color to red, the sampling has been terminated. The cartridge may be removed from the sampling device when the terminated status has been reached. The cartridge is packaged and transferred to a laboratory. The sample collected on the stainless steel electrodes from the cartridge is extracted with buffer and any one or combination of tests are run on the extract. Examples are immunossays for common allergens, β-glucan for active mold, qPCR for common mold species, or assays for mold-generated toxins. The choice of tests is decided by the labortorian, and may also be run sequentially. For example, it might be advantageous to first measure β-glucan, then, if positive, to perform a mold speciation test on the same sample.

It will be appreciated by those skilled in the art that there are many possible modifications to be made to the specific forms of the features and components of the disclosed embodiments while keeping within the spirit of the concepts disclosed herein. Accordingly, no limitations to the specific forms of the embodiments disclosed herein should be read into the claims unless expressly recited in the claims. Although a few embodiments have been described in detail above, other modifications are possible. Other steps may be provided, or steps may be eliminated, from the described methodology, and other components may be added to, or removed from, the described systems. Other embodiments may be within the scope of the following claims.

The foregoing disclosure of specific embodiments is intended to be illustrative of the broad concepts comprehended by the invention.

1. A method for measuring air quality comprising:
operating a real-time air quality sensor to continually measure a parameter of environmental air quality;
automatically determining if the parameter indicates that a select environmental condition has been reached and providing an output signal responsive thereto;
communicating the output signal to an air-quality sampling system;
automatically triggering the air-quality sampling system responsive to the output signal to collect an air sample for a pre-determined time; and
providing laboratory analysis of the collected air sample.

2. The method according to claim 1 wherein laboratory analysis is nucleic acid-based for detection of pathogens.

3. The method according to claim 1 wherein laboratory analysis is immunologically based for detection of toxins or allergens.

4. The method according to claim 1 wherein laboratory analysis is based on limulus amebocyte assays for detection of pyrogens.

5. The system for wirelessly coupling a real time air quality sensor with a sampling system that will collect bio-material over a pre-determined time. for subsequent analysis.

6. The method of claim 1 wherein air quality sensor is a particle sensor. 7 The method of claim 1 wherein air quality sensor is a temperature sensor.

8. The method of claim 1 wherein air quality sensor is a humidity sensor.

9. The method of claim 1 wherein air quality sensor is a volatile organic compound sensor.

10. The method of claim 1 wherein air quality sensor is CO2 sensor

11. The method of claim 1 wherein air quality sensor is a CO sensor.

12. The method of claim 1 wherein air quality sensor is a H2S senso sensor.

13. The method according to claim 1 wherein air quality sensor is a NO sensor.

14. The method according to claim 1 wherein air quality sensor is a NO2 sensor.

15. The method according to claim 1 wherein air quality sensor is a HCHO sensor.

16. The method according to claim 1 wherein air quality sensor is a methane sensor.

17. The method according to claim 1 wherein sampling system is a Burkart sampler.

18. The method according to claim 1 wherein sampling system is an Anderson impactor.

19. The method according to claim 1 wherein communicating the output signal to the air-quality sampling system comprises providing an alert to an operator to activate the air-quality sampling system.
